(19) United States
(12) Patent Application Publication
(10) Pub. No.: US 2024/0268708 A1
(43) Pub. Date: Aug. 15, 2024

(54) EAR-WEARABLE DEVICES FOR FALL PREVENTION USING VIBRATION SENSORY INPUT

(71) Applicant: Starkey Laboratories, Inc., Eden Prairie, MN (US)

(72) Inventors: Andy S. Lin, Chanhassen, MN (US); Sidney A. Higgins, Maple Grove, MN (US); Kyle Olson, St. Louis Park, MN (US); Peter Flanagan, St. Louis Park, MN (US)

(21) Appl. No.: 18/436,646

(22) Filed: Feb. 8, 2024

Related U.S. Application Data
(60) Provisional application No. 63/444,617, filed on Feb. 10, 2023.

(51) Int. Cl. A61B 5/11; A61B 5/00

(52) U.S. Cl. CPC A61B 5/1117; A61B 5/6815

(57) ABSTRACT

Embodiments herein relate to ear-wearable devices that can be used to provide vibration stimulation to device wearers to reduce fall risk. In an embodiment, an ear-wearable device is included having a control circuit, a microphone, and a sensor package. The sensor package can include a touch sensor. The ear-wearable device can further include a vibration generator. The ear-wearable device is configured to evaluate signals from the touch sensor to detect device touching and generate vibrations using the vibration generator when device touching is detected. Other embodiments are also included herein.

This application claims the benefit of U.S. Provisional Application No. 63/444,617, filed Feb. 10, 2023, the content of which is herein incorporated by reference in its entirety.

### FIELD

Embodiments herein relate to ear-wearable devices that can be used to provide vibration stimulation to device wearers to reduce fall risk.

### BACKGROUND

Maintaining postural control and preventing falls are of importance for all individuals, but especially amongst older adults. Falls are a serious public health concern. Falls are the second leading cause of accidental or unintentional injury deaths worldwide. Further, the CDC estimates that one out of every five falls causes an injury, such as broken bones or a head injury. Each year, at least 300,000 individuals are hospitalized for hip fractures with 95% of those caused by falls.

Typical prevention steps for falls can include reviewing medications and their side effects, addressing issues such as postural hypotension, undertaking a proper exercise regimen, checking for and addressing vision problems, checking for and addressing issues with the feet and/or footwear, removing trip hazards, adding grab bars where appropriate, using handrails, and providing proper lighting, amongst others.

### SUMMARY

Embodiments herein relate to ear-wearable devices that can be used to provide vibration stimulation to device wearers to reduce fall risk. In a first aspect, an ear-wearable device can be included having a control circuit, a microphone in electrical communication with the control circuit, and a sensor package. The sensor package can include a touch sensor and can be in electrical communication with the control circuit. The ear-wearable device can also include a vibration generator in electrical communication with the control circuit. The ear-wearable device can be configured to evaluate signals from the touch sensor to detect device touching and generate vibrations using the vibration generator when device touching is detected.

In a second aspect, in addition to one or more of the preceding or following aspects, or in the alternative to some aspects, the sensor package can further include a motion sensor.

In a third aspect, in addition to one or more of the preceding or following aspects, or in the alternative to some aspects, the touch sensor can include at least one selected from the group consisting of a capacitive touch sensor and a resistive touch sensor.

In a fourth aspect, in addition to one or more of the preceding or following aspects, or in the alternative to some aspects, can further include an external housing, wherein the touch sensor can be disposed on the external housing, and wherein the vibration generator can be disposed on the external housing.

In a fifth aspect, in addition to one or more of the preceding or following aspects, or in the alternative to some aspects, the vibration generator and the touch sensor can be co-located on the external housing.

In a sixth aspect, in addition to one or more of the preceding or following aspects, or in the alternative to some aspects, the vibration generator can be configured to deliver vibrations to a device wearer's fingertip contacting the vibration generator.

In a seventh aspect, in addition to one or more of the preceding or following aspects, or in the alternative to some aspects, the vibration generator can be configured to deliver vibrations to a device wearer's external ear or ear canal contacting the vibration generator.

In an eighth aspect, in addition to one or more of the preceding or following aspects, or in the alternative to some aspects, the ear-wearable device can be configured to evaluate signals from the touch sensor to evaluate signals from the touch sensor and generate the vibrations when a predetermined touch pattern can be detected.

In a ninth aspect, in addition to one or more of the preceding or following aspects, or in the alternative to some aspects, the vibrations have a frequency of 60 HZ to 1000 HZ.

In a tenth aspect, in addition to one or more of the preceding or following aspects, or in the alternative to some aspects, the vibrations have a frequency of 150 HZ to 350 HZ.

In an eleventh aspect, in addition to one or more of the preceding or following aspects, or in the alternative to some aspects, the ear-wearable device can be configured to modulate a parameter of the vibrations based on a detected magnitude of device touching pressure.

In a twelfth aspect, in addition to one or more of the preceding or following aspects, or in the alternative to some aspects, the parameter can include at least one selected from the group consisting of vibration frequency, vibration amplitude, and vibration duration.

In a thirteenth aspect, in addition to one or more of the preceding or following aspects, or in the alternative to some aspects, the vibration generator can include at least one selected from the group consisting of a piezoelectric vibration generator and an electromagnetic vibration generator.

In a fourteenth aspect, in addition to one or more of the preceding or following aspects, or in the alternative to some aspects, the piezoelectric vibration generator can include a piezoelectric crystal transducer.

In a fifteenth aspect, in addition to one or more of the preceding or following aspects, or in the alternative to some aspects, the ear-wearable device can include at least one selected from the group consisting of head phones, a RIC hearing aid, and a custom hearing aid.

In a sixteenth aspect, an ear-wearable device can be included having a control circuit, a microphone, and a sensor package. The sensor package can include a touch sensor. The ear-wearable device can be configured to evaluate signals from the touch sensor to detect device touching and send a signal to a separate device to generate vibrations when device touching can be detected.

In a seventeenth aspect, an ear-wearable device can be included having a control circuit, a microphone, and a sensor package. The sensor package can include a motion sensor. The ear-wearable device can include a vibration generator. The ear-wearable device can be configured to evaluate signals from the motion sensor to detect postural instability and generate vibrations using the vibration generator when the postural instability is detected.

In an eighteenth aspect, in addition to one or more of the preceding or following aspects, or in the alternative to some aspects, the ear-wearable device can be configured to generate vibrations using the vibration generator when postural instability exceeds a threshold value.

In a nineteenth aspect, in addition to one or more of the preceding or following aspects, or in the alternative to some aspects, the postural instability can include at least one selected from the group consisting of a leaning angle, a degree of sway, and stumbling.

In a twentieth aspect, in addition to one or more of the preceding or following aspects, or in the alternative to some aspects, the ear-wearable device can further include an external housing, wherein the vibration generator can be disposed on the external housing.

In a twenty-first aspect, in addition to one or more of the preceding or following aspects, or in the alternative to some aspects, the vibration generator can be configured to deliver vibrations to a device wearer's fingertip contacting the vibration generator.

In a twenty-second aspect, in addition to one or more of the preceding or following aspects, or in the alternative to some aspects, the vibration generator can be configured to deliver vibrations to a device wearer's external ear or ear canal contacting the vibration generator.

In a twenty-third aspect, in addition to one or more of the preceding or following aspects, or in the alternative to some aspects, the vibrations can have a frequency of 60 HZ to 1000 HZ.

In a twenty-fourth aspect, in addition to one or more of the preceding or following aspects, or in the alternative to some aspects, the vibrations can have a frequency of 150 HZ to 350 HZ.

In a twenty-fifth aspect, in addition to one or more of the preceding or following aspects, or in the alternative to some aspects, the vibration generator can include at least one selected from the group consisting of a piezoelectric vibration generator and an electromagnetic vibration generator.

In a twenty-sixth aspect, in addition to one or more of the preceding or following aspects, or in the alternative to some aspects, the piezoelectric vibration generator can include a piezoelectric crystal transducer.

In a twenty-seventh aspect, in addition to one or more of the preceding or following aspects, or in the alternative to some aspects, the ear-wearable device can include at least one selected from the group consisting of head phones, a RIC hearing aid, and a custom hearing aid.

In a twenty-eighth aspect, in addition to one or more of the preceding or following aspects, or in the alternative to some aspects, the ear-wearable device can be configured to modulate a parameter of the vibrations based on a degree of detected postural instability.

In a twenty-ninth aspect, in addition to one or more of the preceding or following aspects, or in the alternative to some aspects, the modulated parameter can include at least one selected from the group consisting of vibration frequency, vibration amplitude, and vibration duration.

In a thirtieth aspect, an ear-wearable device can be included having a control circuit, a microphone, and a sensor package. The sensor package can include a motion sensor. The ear-wearable device can be configured to evaluate signals from the motion sensor to detect postural instability and send a signal to a separate device to generate vibrations when postural instability can be detected.

In a thirty-first aspect, an ear-wearable device can be included having a control circuit, a microphone, and a sensor package. The sensor package can include a motion sensor. The ear-wearable device can include a vibration generator. The ear-wearable device can be configured to evaluate signals from the microphone to detect an initiation sound and generate vibrations using the vibration generator when the initiation sound can be detected.

In a thirty-second aspect, in addition to one or more of the preceding or following aspects, or in the alternative to some aspects, the initiation sound can include at least one selected from the group consisting of a verbal command, an identified keyword or phrase, and an instability utterance.

In a thirty-third aspect, in addition to one or more of the preceding or following aspects, or in the alternative to some aspects, the ear-wearable device can further include a housing, wherein the vibration generator can be disposed on the housing.

In a thirty-fourth aspect, in addition to one or more of the preceding or following aspects, or in the alternative to some aspects, the vibration generator can be configured to deliver vibrations to a device wearer's fingertip contacting the vibration generator.

In a thirty-fifth aspect, in addition to one or more of the preceding or following aspects, or in the alternative to some aspects, the vibration generator can be configured to deliver vibrations to a device wearer's external ear or ear canal contacting the vibration generator.

In a thirty-sixth aspect, in addition to one or more of the preceding or following aspects, or in the alternative to some aspects, the vibrations can have a frequency of 60 HZ to 1000 HZ.

In a thirty-seventh aspect, in addition to one or more of the preceding or following aspects, or in the alternative to some aspects, the vibrations can have a frequency of 150 HZ to 350 HZ.

In a thirty-eighth aspect, in addition to one or more of the preceding or following aspects, or in the alternative to some aspects, the vibration generator can include at least one selected from the group consisting of a piezoelectric vibration generator and an electromagnetic vibration generator.

In a thirty-ninth aspect, in addition to one or more of the preceding or following aspects, or in the alternative to some aspects, the piezoelectric vibration generator can include a piezoelectric crystal transducer.

In a fortieth aspect, in addition to one or more of the preceding or following aspects, or in the alternative to some aspects, the ear-wearable device can include at least one selected from the group consisting of head phones, a RIC hearing aid, and a custom hearing aid.

In a forty-first aspect, an ear-wearable device can be included having a control circuit, a microphone, and a sensor package. The sensor package can include a motion sensor. The ear-wearable device can be configured to evaluate signals from the microphone to detect an initiation sound and send a signal to a separate device to generate vibrations when the initiation sound can be detected.

In a forty-second aspect, a method of providing vibration stimulation to an ear-wearable device wearer can be included. The method can include evaluating signals from a sensor or a microphone to detect a trigger condition and generating vibrations using a vibration generator when the trigger condition can be detected.

In a forty-third aspect, in addition to one or more of the preceding or following aspects, or in the alternative to some aspects, the sensor can include a touch sensor. The trigger condition can include the device wearer touching the touch sensor.

In a forty-fourth aspect, in addition to one or more of the preceding or following aspects, or in the alternative to some aspects, the method can further include evaluating signals from a touch sensor to evaluate signals from a touch sensor and generating the vibrations when a predetermined touch pattern can be detected.

In a forty-fifth aspect, in addition to one or more of the preceding or following aspects, or in the alternative to some aspects, the method can further include modulating a parameter of the vibrations based on a detected magnitude of device touching pressure.

In a forty-sixth aspect, in addition to one or more of the preceding or following aspects, or in the alternative to some aspects, the method can further include delivering vibrations to a device wearer's fingertip contacting the vibration generator.

In a forty-seventh aspect, in addition to one or more of the preceding or following aspects, or in the alternative to some aspects, the method can further include delivering vibrations to a device wearer's external ear or ear canal contacting the vibration generator.

In a forty-eighth aspect, in addition to one or more of the preceding or following aspects, or in the alternative to some aspects, the trigger condition can include an initiation sound.

In a forty-ninth aspect, in addition to one or more of the preceding or following aspects, or in the alternative to some aspects, the initiation sound can include at least one selected from the group consisting of a verbal command, an identified keyword or phrase, and an instability utterance.

In a fiftieth aspect, in addition to one or more of the preceding or following aspects, or in the alternative to some aspects, the trigger condition can include a motion sensor signal pattern.

In a fifty-first aspect, in addition to one or more of the preceding or following aspects, or in the alternative to some aspects, the motion sensor signal pattern can be indicative of postural instability.

In a fifty-second aspect, in addition to one or more of the preceding or following aspects, or in the alternative to some aspects, the vibrations have a frequency of 60 HZ to 1000 HZ.

In a fifty-third aspect, in addition to one or more of the preceding or following aspects, or in the alternative to some aspects, the vibrations have a frequency of 150 HZ to 350 HZ.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of a device wearer with ear-wearable devices in accordance with various embodiments herein.

FIG. 2 is a flow chart of operations in accordance with various embodiments herein.

FIG. 3 is a schematic view of a device wearer with ear-wearable devices in accordance with various embodiments herein.

FIG. 4 is a schematic view of an ear-wearable device in accordance with various embodiments herein.

FIG. 5 is a schematic view of an ear-wearable device within an ear of a device wearer in accordance with various embodiments herein.

FIG. 6 is a schematic view of an ear-wearable device in accordance with various embodiments herein.

FIG. 7 is a schematic view of a device wearer with ear-wearable devices in accordance with various embodiments herein.

FIG. 8 is a schematic view of a device wearer with ear-wearable devices in accordance with various embodiments herein.

FIG. 9 is a schematic view of an accessory device in accordance with various embodiments herein.

FIG. 10 is a schematic view of a system environment in accordance with various embodiments herein.

FIG. 11 is a block diagram of components of an ear-wearable device in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

As referenced above, falls are a serious public health concern. Typical prevention steps for falls are important to take yet may still leave an individual vulnerable for a fall. As such, there is a need for further approaches or interventions to prevent falls.

Embodiments herein include ear-wearable devices that can used to help prevent falls by providing vibration stimulation to individuals to reduce their fall risk. As an example, an ear-wearable device herein can include a control circuit, a microphone, and a sensor package. The sensor package can include a touch sensor. The ear-wearable device can also include a vibration generator. The ear-wearable device can be configured to evaluate signals from the touch sensor to detect device touching and generate vibrations using the vibration generator when device touching is detected. Alternatively, instead of generating vibrations with the ear-wearable device or in addition to that, a signal can be sent to a separate device to generate vibrations.

While the use of a touch sensor can provide for the device wearer to initiate vibration stimulation as desired, in some embodiments herein vibration stimulation can be initiated in other ways. For example, the ear-wearable device can include a motion sensor to be able to detect postural instability. When detected, the ear-wearable device can generate vibrations using a vibration generator and/or send a signal to a separate device to generate vibrations.

As a further example, in some embodiments ear-wearable devices herein can be configured to evaluate signals from a microphone to detect an initiation sound (such as a verbal command, an identified keyword or phrase, an instability utterance, etc.). When the initiation sound is detected, the ear-wearable device can generate vibrations using the vibration generator.

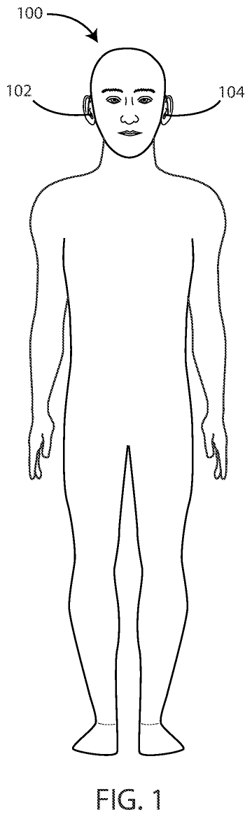

Referring now to FIG. 1, a schematic view of a device wearer 100 with ear-wearable devices is shown in accordance with various embodiments herein. In specific, FIG. 1 shows the device wearer 100 with a first ear-wearable device 102 and a second ear wearable device 104. One or both of the ear-wearable devices can determine when to begin vibration stimulation and then administer the same. The vibration stimulation can be provided to a skin surface of the device wearer 100 such as on their fingers, hands, ears, ear canal, etc. The vibration stimulation can be effective to prevent falls from occurring.

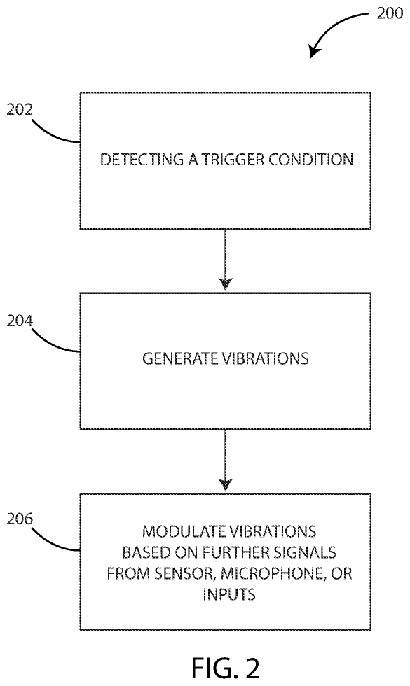

Referring now to FIG. 2, a flow chart of operations is shown in accordance with various embodiments herein. In this example, the set of operations 200 for providing vibration stimulation to an ear-wearable device wearer includes detecting a trigger condition 202. Trigger conditions can include evaluating signals from a touch sensor to detect device touching (such as the device wearer touching the ear-wearable device at a certain place on the device with their finger). Trigger conditions can also include detecting postural instability (such as a degree of sway indicating a danger of falling) with a motion sensor and/or other sensors. Trigger conditions can also include detecting an initiation sound with a microphone. Trigger conditions can also include an input received from the device wearer, a third party (such as a care provider), and/or a separate device or system. Various other trigger conditions are also contemplated herein and described below.

The set of operations 200 can further include generating vibrations 204 when the trigger condition is detected. Details of exemplary vibrations and parameters thereof are provided in greater detail below. The ear-wearable device can generate vibrations using a vibration generator. In some cases, a vibration generator can be integrated into the ear-wearable device. However, in some embodiments, the vibration generator can be part of a separate device.

In some scenarios, the set of operations 200 can further include modulating a parameter of the vibrations 206, such as modulating a parameter based on signals from a sensor, a microphone, or inputs from the device wearer, a third party, or another system or device. Exemplary parameters of vibration are described in greater detail below but can include vibration frequency, vibration amplitude, vibration duration, and the like.

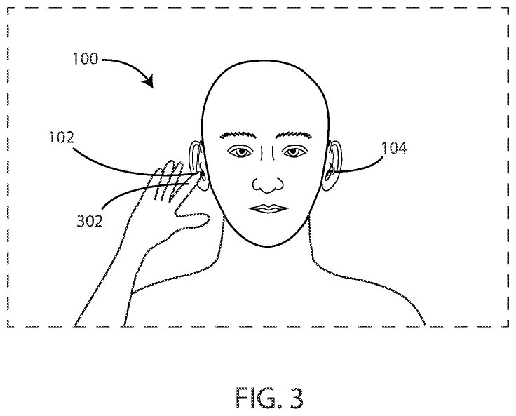

In various embodiments, a vibration generator herein can specifically be configured to deliver vibrations to a device wearer's fingertip contacting the vibration generator. For example, referring now to FIG. 3, a schematic view of a device wearer 100 with car-wearable devices 102, 104 is shown in accordance with various embodiments herein. In this example, the device wearer 100 has reached up and is pressing their finger 302 against the car-wearable device 102. Contact of their finger 302 with the car-wearable device 102 can serve as the trigger condition to turn on the delivery of vibrations as well as provide contact with a vibration generator for the device wearer 100 to receive the generated vibrations. In some embodiments, however, only one of these functions may occur based on contact of their finger 302 (or another body part) with the car-wearable device 102. For example, in some cases contact of their finger 302 with the car-wearable device 102 can serve as a trigger condition to turn on the delivery of vibrations, but vibrations are not received through their finger 302. Conversely, in some cases, vibrations may be received through their finger 302, but a trigger condition other than contact of the finger 302 with the car-wearable device 102 is utilized.

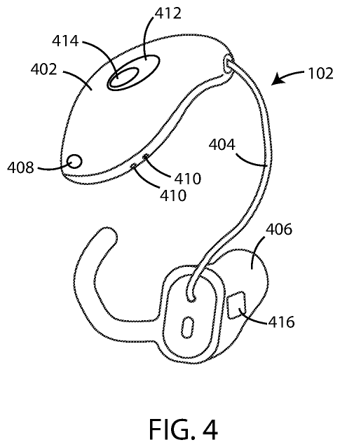

Ear-wearable devices herein can take on many different forms and can include a number of different components. Referring now to FIG. 4, a schematic view of an exemplary car-wearable device 102 is shown in accordance with various embodiments herein. The car-wearable device 102 includes an external housing 402. Various components of the ear-wearable device 102 can be housed within the external housing 402. The car-wearable device 102 also includes a cable 404. The car-wearable device 102 also includes a receiver 406. The receiver 406 can include a transducer or speaker to generate sound.

In some embodiments, the car-wearable device 102 also includes a button 408 or other physical input device. The button 408 can be used by the device wearer to provide one or more inputs to the ear-wearable device 102. For example, in some embodiments, the button 408 can be pressed and serve as a trigger condition to start the delivery of vibrations. In some embodiments, the car-wearable device 102 also includes electrical contacts 410, which can be used for recharging a battery in the car-wearable device 102.

The ear-wearable device 102 also includes a vibration generator 412. In some embodiments, the car-wearable device 102 includes an internal vibration generator 416. In various embodiments, the vibration generator 412 can be disposed on or inside of an external housing 402. In various embodiments, the vibration generator 412 can be configured to deliver vibrations to a device wearer's fingertip in contact with the vibration generator 412. In various embodiments, the vibration generator 412 can be configured to deliver vibrations to a device wearer's external car or car canal in contact with a vibration generator 412. In various embodiments, the vibration generator 412 can include at least one selected from the group consisting of a piezoelectric vibration generator and an electromagnetic vibration generator. In various embodiments, the piezoelectric vibration generator 412 can include a piezoelectric crystal transducer.

A sensor package of the car-wearable device 102 can include a touch sensor 414. In various embodiments, the touch sensor 414 can include at least one selected from the group consisting of a capacitive touch sensor and a resistive touch sensor. In various embodiments, the touch sensor 414 is disposed on the external housing 402. The touch sensor 414 can be used to detect touching based on evaluating the signal(s) from the touch sensor 414. For example, touching a resistive touch sensor can make the resistance or impedance of the sensor change significantly.

In various embodiments, the car-wearable device 102 can be configured to evaluate signals from the touch sensor 414 and immediately begin to generate vibrations when touching is detected. However, in various embodiments, the car-wearable device 102 can be configured to evaluate signals from the touch sensor 414 and generate the vibrations when a predetermined touch pattern is be detected. The predetermined touch pattern can include various patterns such as a series of touches (or taps). In some embodiments, the predetermined touch pattern can include a touch lasting a predetermined time period, such as a continuous touch lasting more than 1, 2, 3, or 5 seconds or longer.

In various embodiments, the vibration generator 412 and a touch sensor 414 are co-located on an external housing 402. In this configuration, when a device wearer touches the touch sensor 414, they also receive vibrations with the same part of the finger, hand or other body part in contact with the touch sensor 414 because the vibration generator 412 is collocated therewith, such as underneath the touch sensor 414 or immediately adjacent to the touch sensor 414.

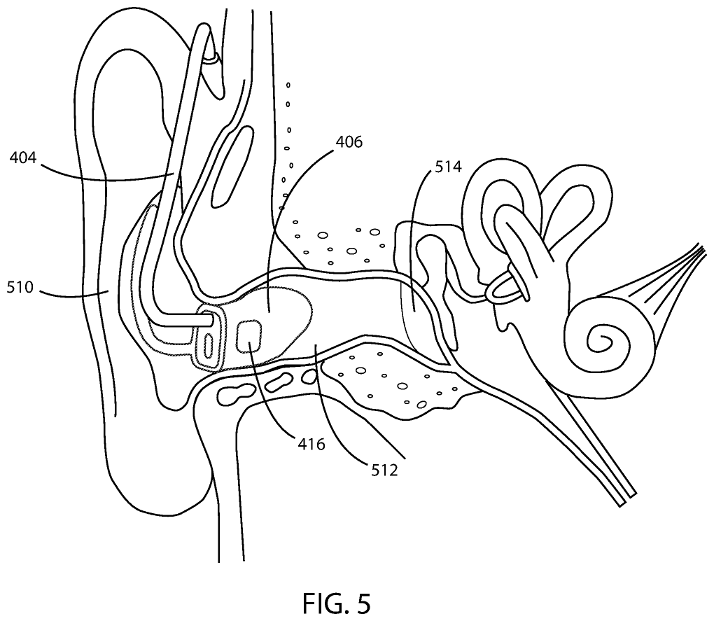

Referring now to FIG. 5, a schematic view of an ear-wearable device 102 within an ear of a device wearer 100 is shown in accordance with various embodiments herein. The ear includes a pinna 510, an ear canal 512, and a tympanic membrane 514. The ear-wearable device includes a cable 404 and a receiver 406. In various embodiments, the vibration generator 412 can be configured to deliver vibrations to a device wearer's external ear or ear canal 512 contacting the vibration generator 412.

In various embodiments, the ear-wearable device 102 can include at least one of headphones, a RIC hearing aid, and a custom hearing aid. However, many different types of devices are contemplated herein including, but not limited to, behind-the-ear (BTE), in-the ear (ITE), in-the-canal (ITC), invisible-in-canal (IIC), receiver-in-canal (RIC), receiver in-the-ear (RITE) and completely-in-the-canal (CIC) type hearing assistance devices.

The term “ear-wearable device” shall also refer to devices that can produce optimized or processed sound for persons with normal hearing. Ear-wearable devices herein can include hearing assistance devices. In some embodiments, the ear-wearable device can be a hearing aid falling under 21 C.F.R. § 801.420. In another example, the ear-wearable device can include one or more Personal Sound Amplification Products (PSAPs). In another example, the ear-wearable device can include one or more cochlear implants, cochlear implant magnets, cochlear implant transducers, and cochlear implant processors. In another example, the ear-wearable device can include one or more “hearable” devices that provide various types of functionality. In other examples, ear-wearable devices can include other types of devices that are wearable in, on, or in the vicinity of the user's ears. In other examples, ear-wearable devices can include other types of devices that are implanted or otherwise osseointegrated with the user's skull; wherein the device is able to facilitate stimulation of the wearer's ears via the bone conduction pathway.

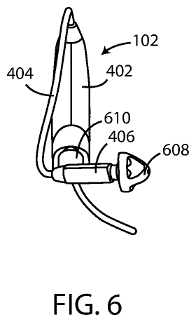

Referring now to FIG. 6, a schematic view of another type of ear-wearable device 102 is shown in accordance with various embodiments herein. As before, the ear-wearable device 102 includes an external housing 402, a cable 404, and a receiver 406.

However, in this configuration, the ear-wearable device 102 also includes an ear bud 608. The ear-wearable device 102 can also include a battery compartment 610.

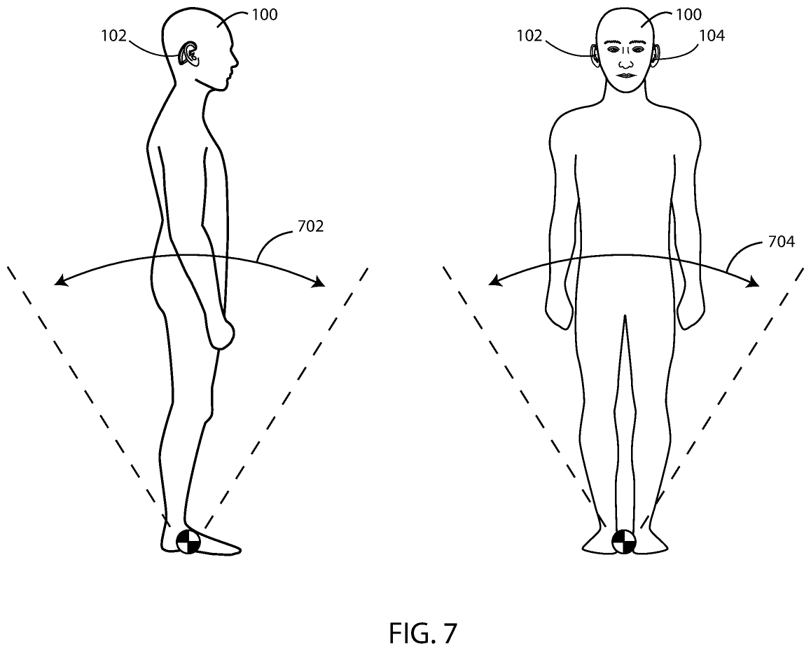

As referenced above, devices herein can begin providing vibration stimulation to an ear-wearable device wearer after detecting a trigger condition. Trigger conditions can specifically include detecting postural instability with a motion sensor and/or other sensors. Referring now to FIG. 7, a schematic view of a device wearer 100 with ear-wearable devices 102, 104 is shown in accordance with various embodiments herein. Aspects of postural instability can specifically include sway. For example, aspects of postural instability can include forward and backward sway 702 as well as lateral sway 704. The sensor package of the ear-wearable device can include a motion sensor to detect sway or other aspects of postural instability. The ear-wearable device 102 can be configured to evaluate signals from the motion sensor to detect postural instability and can be configured to generate vibrations using a vibration generator when the postural instability is detected.

In various embodiments, the ear-wearable device 102 can be configured to generate vibrations using the vibration generator when postural instability exceeds a threshold value. For example, if the amount of postural sway (as is detectable from motion sensor signals) crosses a threshold value, then the ear-wearable device can treat that as a trigger condition and initiate generation of vibrations. As another example, if the leaning angle crosses a threshold value (as is detectable from an IMU and/or a gyroscope signal), then the ear-wearable device can treat that as a trigger condition and initiate generation of vibrations.

In various embodiments, the ear-wearable device 102 can be configured to modulate a parameter of the vibrations based on a degree of detected postural instability. For example, the amplitude of the vibrations can be increased or decreased based on the degree of detected postural instability. As another example, the frequency of the vibrations can be increased or decreased based on the degree of detected postural instability.

It will be appreciated that various other aspects of postural instability can be evaluated by the device beyond sway. For example, aspects of postural instability evaluated by devices herein can include a leaning angle, a degree of sway, postural asymmetries, smoothness of postural movements, stumbling, and the like.

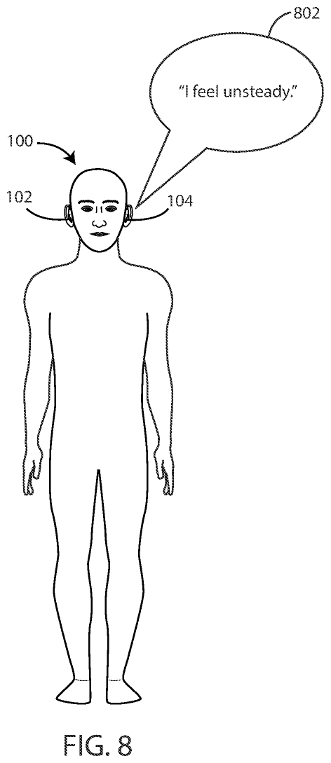

Trigger conditions herein can also include the detection of an initiation sound. Referring now to FIG. 8, a schematic view of a device wearer 100 with car-wearable devices 102, 104 is shown in accordance with various embodiments herein. In this example, a trigger condition (or trigger input) can be an initiation sound 802. The range of possible initiation sounds 802 can vary. In some embodiments, the initiation sound 802 can include at least one selected from the group consisting of a verbal command, an identified keyword or phrase, and an instability utterance. Exemplary verbal commands can include, but are not limited to, those such as “start vibrations”, “start stimulation”, “help steady me”, and the like. Exemplary keywords or phrases can include, but are not limited to, “I feel unsteady”, “I'm feeling dizzy”, “I'm losing my balance”, and the like. Exemplary instability utterances can include, but are not limited to, “whoa” and other utterances indicative of an imminent fall.

In many embodiments herein, vibrations can be generated and/or delivered to the device wearer directly from the car-wearable device itself. However, in some embodiments, the car-wearable device can send a command or instruction to a separate device or system in order to start delivering vibrations to the device wearer. For example, in some embodiments, the car-wearable device can send a command or instruction to an accessory device to deliver vibrations to the device wearer.

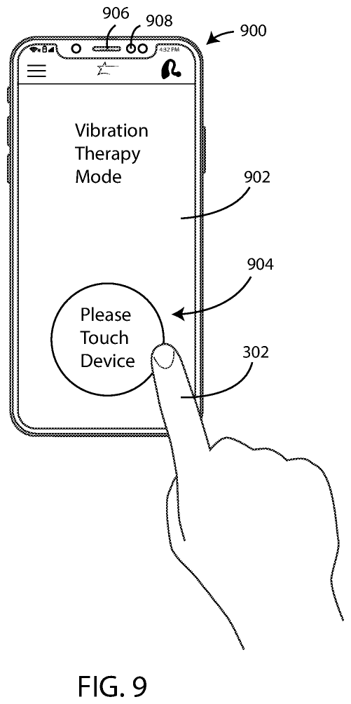

Referring now to FIG. 9, a schematic view of an accessory device 900 is shown in accordance with various embodiments herein. The accessory device 900 includes a display screen 902, a speaker 906, and a camera 908. The display screen 902 includes an instruction 904 displayed thercon. The device wearer's finger 302 can contact the accessory device 900. The accessory device 900 can be configured to receive a command from an car-wearable device to initiate vibration stimulation. The accessory device 900 can include a vibration generator and can generate vibrations received by the device wearer through their finger 302 and/or another body part in contact therewith.

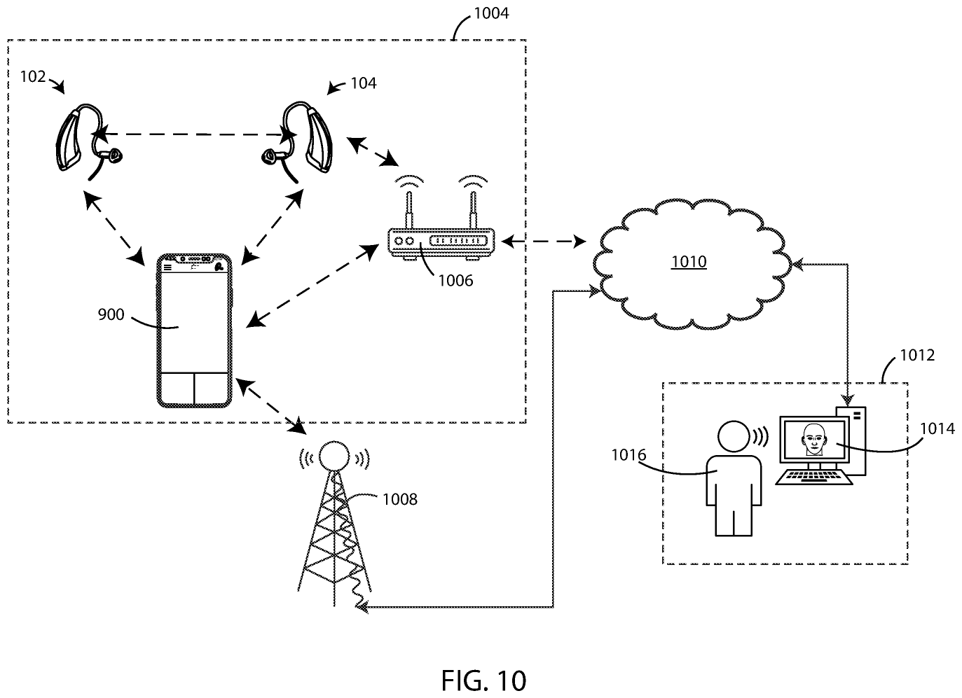

Systems herein can include various separate devices and can transfer data and/or signals between the same. Referring now to FIG. 10, a schematic view of a system environment is shown in accordance with various embodiments herein. FIG. 10 shows a first car-wearable device 102 and a second ear wearable device 104. FIG. 10 also shows an accessory device 900. The accessory device 900 can be used to detect trigger conditions and/or can be used to generate and/or deliver vibrations to the device wearer. FIG. 10 illustrates a local environment 1004 with a router 1006. Data communications can be facilitated through the router 1006 and/or through a cell tower 1008 or another type of data communication antenna or receiver. Data communications can pass through the cloud 1010 or another data network. The cloud 1010 can include various computing resources to execute operations herein including, but not limited to, servers (real or virtual), databases, and the like. FIG. 10 also shows a remote environment 1012. The remote environment 1012 can include a remote computing device 1014 or other access device and can be used by a care provider 1016.

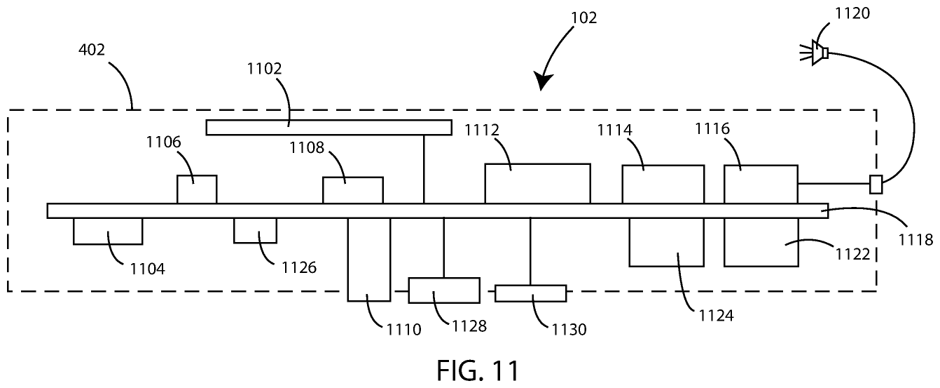

Referring now to FIG. 11, a schematic block diagram of components of an exemplary car-wearable device is shown in accordance with various embodiments herein. The car-wearable device 102 shown in FIG. 11 includes several components electrically connected to a flexible mother circuit 1118 (e.g., flexible mother board) which is disposed within housing 402. A power supply circuit 1104 can include a battery and can be electrically connected to the flexible mother circuit 1118 and provides power to the various components of the car-wearable device 102. One or more microphones 1106 are electrically connected to the flexible mother circuit 1118, which provides electrical communication between the microphones 1106 and a digital signal processor (DSP) 1112. Among other components, the DSP 1112 incorporates or is coupled to audio signal processing circuitry configured to implement various functions described herein. A sensor package 1114 can be coupled to the DSP 1112 via the flexible mother circuit 1118. The sensor package 1114 can include one or more different specific types of sensors such as those described in greater detail below. One or more user switches 1110 (e.g., on/off, volume, mic directional settings) are electrically coupled to the DSP 1112 via the flexible mother circuit 1118.

An audio output device 1116 is electrically connected to the DSP 1112 via the flexible mother circuit 1118. In some embodiments, the audio output device 1116 comprises an electroacoustic transducer or speaker (coupled to an amplifier). In other embodiments, the audio output device 1116 comprises an amplifier coupled to an external receiver 1120 adapted for positioning within an ear of a wearer. The external receiver 1120 can include an electroacoustic transducer, speaker, or loudspeaker. The ear-wearable device 102 may incorporate a communication device 1108 coupled to the flexible mother circuit 1118 and to an antenna 1102 directly or indirectly via the flexible mother circuit 1118. The communication device 1108 can be a BLUETOOTH® transceiver, such as a BLE (BLUETOOTH® low energy) transceiver or other transceiver(s) (e.g., an IEEE 802.11 compliant device). The communication device 1108 can be configured to communicate with one or more external devices, such as those discussed previously, in accordance with various embodiments. In various embodiments, the communication device 1108 can be configured to communicate with an external visual display device such as a smart phone, a video display screen, a tablet, a computer, or the like.

In various embodiments, the ear-wearable device 102 can also include a control circuit 1122 and a memory storage device 1124. The control circuit 1122 can be in electrical communication with other components of the device. In some embodiments, a clock circuit 1126 can be in electrical communication with the control circuit. The control circuit 1122 can execute various operations, such as those described herein. The control circuit 1122 can include various components including, but not limited to, a microprocessor, a microcontroller, an FPGA (field-programmable gate array) processing device, an ASIC (application specific integrated circuit), or the like. The memory storage device 1124 can include both volatile and non-volatile memory. The memory storage device 1124 can include ROM, RAM, flash memory, EEPROM, SSD devices, NAND chips, and the like. The memory storage device 1124 can be used to store data from sensors as described herein and/or processed data generated using data from sensors as described herein.

In various embodiments, the ear-wearable device 102 can also include a vibration generator 1128. Various types of vibration generators can be used herein including, but not limited to, piezoelectric vibration generators and electromagnetic vibration generators. An exemplary piezoelectric vibration generator herein can be, for example, a piezoelectric crystal transducer.

In various embodiments, the ear-wearable device 102 can also include a touch sensor 1130. Various types of touch sensors can be used including, but not limited to, capacitive touch sensors and resistive touch sensors.

It will be appreciated that various of the components described in FIG. 11 can be associated with separate devices and/or accessory devices to the ear-wearable device. By way of example, microphones can be associated with separate devices and/or accessory devices. Similarly, audio output devices and/or vibration generators can be associated with separate devices and/or accessory devices to the ear-wearable device.

### Vibrations and Parameters Thereof

Vibrations herein can be provided at various frequencies. The vibrations can be provided at a frequency or range of frequencies that is effective to reduce fall risk. In some embodiments, the frequency can be greater than or equal to 50 HZ, 100 HZ, 150 HZ, 200 HZ, 250 HZ, 300 HZ, 350 HZ, 400 HZ, 450 HZ, or 500 HZ. In some embodiments, the frequency can be less than or equal to 1000 HZ, 950 HZ, 900 HZ, 850 HZ, 800 HZ, 750 HZ, 700 HZ, 650 HZ, 600 HZ, 550 HZ, 500 HZ, 450 HZ, 400 HZ, 350 HZ, or 300 HZ. In some embodiments, the frequency or frequencies can fall within a range between any of the foregoing. In various embodiments, the vibrations have a frequency of 60 HZ to 1000 HZ. In various embodiments, the vibrations have a frequency of 150 HZ to 350 HZ. In some embodiments, the frequency or frequencies can be substantially constant over time. In other embodiments the frequency or frequencies can vary over time.

Vibrations herein can be provided at various amplitudes. In some embodiments, the vibrations can be provided at a substantially constant amplitude over time. In other embodiments, the vibrations can be provided at an amplitude that varies over time. In some embodiments, vibration amplitudes can be greater than or equal to 0.001, 0.01, 0.1, 0.5, 0.75, or 1 mm or an amplitude falling within a range between any of the foregoing. In various embodiments herein, if or when amplitude is modulated it can be done as a relative amount with respect to a baseline amplitude value. As such, in some embodiments, amplitude can be changed by increasing it by 10, 25, 50, 100, 200, 500, or 1000 percent or more, or an amount falling within a range between any of the foregoing. In some embodiments, amplitude can be changed by decreasing it by 10, 20, 30, 40, 50, 60, 70, 80, 90, 95, or 99 percent, or an amount falling within a range between any of the foregoing.

In some embodiments, vibrations can be provided substantially constantly over a time period or vibration duration. In other embodiments, vibrations can be provided intermittently over a time period, such as with a series of discrete vibration periods. The time period over which vibrations are provided can be predetermined or set by a system user. In some embodiments, the time period can be greater than or equal to 0, 50, 100, 150, 200, 250, 300, 350, 400, 450, 500, 550, or 600 seconds, or can be an amount of time falling within a range between any of the foregoing.

In various embodiments, the ear-wearable device 102 can be configured to modulate a parameter (such as any described herein) of the vibrations in certain scenarios. For example, the ear-wearable device 102 can be configured to modulate a parameter of the vibrations based on a detected magnitude of device touching pressure. As another example, the ear-wearable device 102 can be configured to modulate a parameter of the vibrations based on a detected magnitude of postural instability.

### Postural Instability Detection

As described above, in various embodiments the system can initiate the provision of vibrations when postural instability is detected. Aspects of postural instability evaluated by devices herein can include one or more of a leaning angle, a degree of sway, postural asymmetries, smoothness of postural movements, stumbling, and the like. Detection of postural instability and/or aspects thereof can be performed in various ways. In various embodiments herein, a device or a system can be used to detect a pattern or patterns (such as patterns of data from sensors) indicative of postural instability and/or postural instability of a certain magnitude. Such patterns can be detected in various ways. Some techniques are described elsewhere herein, but some further examples will now be described.

As merely one example, one or more sensors can be operatively connected to a controller (such as the control circuit described in FIG. 11) or another processing resource (such as a processor of another device or a processing resource in the cloud). The controller or other processing resource can be adapted to receive data representative of a characteristic of the subject from one or more of the sensors and/or determine statistics of the subject over a monitoring time period based upon the data received from the sensor. As used herein, the term “data” can include a single datum or a plurality of data values or statistics. The term “statistics” can include any appropriate mathematical calculation or metric relative to data interpretation, e.g., probability, confidence interval, distribution, range, or the like. Further, as used herein, the term “monitoring time period” means a period of time over which characteristics of the subject are measured and statistics are determined. The monitoring time period can be any suitable length of time, e.g., 0.1 seconds, 1 second, 5 seconds, 10 seconds, 30 seconds, 1 minute, 10 minutes, 30 minutes, 1 hour, 1 day, 1 week, etc., or a range of time between any of the foregoing time periods.

Any suitable technique or techniques can be utilized to determine statistics for the various data from the sensors, e.g., direct statistical analyses of time series data from the sensors, differential statistics, comparisons to baseline or statistical models of similar data, etc. Such techniques can be general or individual-specific and represent long-term or short-term behavior. These techniques could include standard pattern classification methods such as Gaussian mixture models, clustering as well as Bayesian approaches, neural network models and deep learning, and the like.

Further, in some embodiments, the controller can be adapted to compare data, data features, and/or statistics against various other patterns or templates, which could be prerecorded patterns (baseline patterns) of the particular individual wearing an car-wearable device herein, prerecorded patterns (group baseline patterns) of a group of individuals wearing car-wearable devices herein, one or more predetermined patterns that serve as positive example patterns (such as patterns indicative of postural instability), negative example patterns, or the like. As merely one scenario, if a pattern is detected in an individual that exhibits similarity crossing a threshold value to a positive example pattern or substantial similarity to that pattern, then that can be taken as an indication of the occurrence of postural instability associated with the positive example pattern. Positive and/or negative example patterns can be stored or accessed for use covering those items to be detected in accordance with embodiments herein including, but not limited to, postural instability and events impacting postural instability. In some embodiments, patterns or templates can be indexed for severity of postural instability. In this manner, the severity of the postural instability can be characterized. However, in some embodiments, severity can simply be characterized by the magnitude of motion sensor or other sensor signals. In some embodiments, a trend in severity over time can be calculated.

Similarity and dissimilarity can be measured directly via standard statistical metrics such normalized Z-score, or similar multidimensional distance measures (e.g. Mahalanobis or Bhattacharyya distance metrics), or through similarities of modeled data and machine learning. These techniques can include standard pattern classification methods such as Gaussian mixture models, clustering as well as Bayesian approaches, neural network models, and deep learning.

As used herein the term “substantially similar” means that, upon comparison, the sensor data are congruent or have statistics fitting the same statistical model, each with an acceptable degree of confidence. The threshold for the acceptability of a confidence statistic may vary depending upon the subject, sensor, sensor arrangement, type of data, context, condition, etc.

The statistics associated with the loss of balance event of an individual over the monitoring time period, can be determined by utilizing any suitable technique or techniques, e.g., standard pattern classification methods such as Gaussian mixture models, clustering, hidden Markov models, as well as Bayesian approaches, neural network models, and deep learning.

Various embodiments herein specifically include the application of a machine learning classification model. In various embodiments, the ear-wearable system can be configured to periodically update the machine learning classification model based on indicators of postural instability experienced by the device wearer.

In some embodiments, a training set of data can be used in order to generate a machine learning classification model. The input data can include motion sensor data and/or other sensor data as described herein as tagged/labeled with binary and/or non-binary classifications of postural instability. Binary classification approaches can utilize techniques including, but not limited to, logistic regression, k-nearest neighbors, decision trees, support vector machine approaches, naive Bayes techniques, and the like. Multi-class classification approaches (e.g., for non-binary classifications of triggers and/or allergic reactions) can include k-nearest neighbors, decision trees, naive Bayes approaches, random forest approaches, and gradient boosting approaches amongst others. In various embodiments, the ear-wearable system is configured to execute operations to generate or update the machine learning model on the ear-wearable device itself. In some embodiments, the ear-wearable system may convey data to another device such as an accessory device or a cloud computing resource in order to execute operations to generate or update a machine learning model herein. In various embodiments, the ear-wearable system is configured to weight certain possible detected indicators of postural instability in the machine learning classification model more heavily based on derived correlations specific for the individual as described elsewhere herein.

### Sensors

Ear-wearable devices herein can include one or more sensor packages (including one or more discrete or integrated sensors) to provide data. The sensor package can comprise one or a multiplicity of sensors. In some embodiments, the sensor packages can include one or more motion sensors (or movement sensors) amongst other types of sensors. Motion sensors herein can include inertial measurement units (IMU), accelerometers, gyroscopes, barometers, altimeters, and the like. The IMU can be of a type disclosed in commonly owned U.S. Pat. No. 9,848,273, which is incorporated herein by reference. In some embodiments, electromagnetic communication radios or electromagnetic field sensors (e.g., telecoil, NFMI, TMR, GMR, etc.) sensors may be used to detect motion or changes in position. In various embodiments, the sensor package can include a magnetometer. In some embodiments, biometric sensors may be used to detect body motions or physical activity. Motions sensors can be used to track movement of a patient in accordance with various embodiments herein.

In some embodiments, the motion sensors can be disposed in a fixed position with respect to the head of a patient, such as worn on or near the head or ears. In some embodiments, the operatively connected motion sensors can be worn on or near another part of the body such as on a wrist, arm, or leg of the patient.

According to various embodiments, the sensor package can include one or more of an IMU, and accelerometer (3, 6, or 9 axis), a gyroscope, a barometer, an altimeter, a magnetometer, a magnetic sensor, an eye movement sensor, a pressure sensor, an acoustic sensor, a telecoil, a heart rate sensor, a global positioning system (GPS), a temperature sensor, a blood pressure sensor, an oxygen saturation sensor, an optical sensor, a blood glucose sensor (optical or otherwise), a galvanic skin response sensor, a cortisol level sensor (optical or otherwise), a microphone, acoustic sensor, an electrocardiogram (ECG) sensor, electroencephalography (EEG) sensor which can be a neurological sensor, eye movement sensor (e.g., electrooculogram (EOG) sensor), myographic potential electrode sensor (EMG), a heart rate monitor, a pulse oximeter or oxygen saturation sensor (SpO2), a wireless radio antenna, blood perfusion sensor, hydrometer, sweat sensor, cerumen sensor, air quality sensor, pupillometry sensor, cortisol level sensor, hematocrit sensor, light sensor, image sensor, and the like. In various embodiments, the sensor package can include a touch sensor.

In some embodiments, the sensor package can be part of an ear-wearable device. However, in some embodiments, the sensor packages can include one or more additional sensors that are external to an ear-wearable device. For example, various of the sensors described above can be part of a wrist-worn or ankle-worn sensor package, or a sensor package supported by a chest strap. In some embodiments, sensors herein can be disposable sensors that are adhered to the device wearer (“adhesive sensors”) and that provide data to the ear-wearable device or another component of the system.

Data produced by the sensor(s) of the sensor package can be operated on by a processor of the device or system.

As used herein the term “inertial measurement unit” or “IMU” shall refer to an electronic device that can generate signals related to a body's specific force and/or angular rate. IMUs herein can include one or more accelerometers (3, 6, or 9 axis) to detect linear acceleration and a gyroscope to detect rotational rate. In some embodiments, an IMU can also include a magnetometer to detect a magnetic field.

The eye movement sensor may be, for example, an electrooculographic (EOG) sensor, such as an EOG sensor disclosed in commonly owned U.S. Pat. No. 9,167,356, which is incorporated herein by reference. The pressure sensor can be, for example, a MEMS-based pressure sensor, a piezo-resistive pressure sensor, a flexion sensor, a strain sensor, a diaphragm-type sensor and the like.

The temperature sensor can be, for example, a thermistor (thermally sensitive resistor), a resistance temperature detector, a thermocouple, a semiconductor-based sensor, an infrared sensor, or the like.

The blood pressure sensor can be, for example, a pressure sensor. The heart rate sensor can be, for example, an electrical signal sensor, an acoustic sensor, a pressure sensor, an infrared sensor, an optical sensor, or the like.

The oxygen saturation sensor (such as a blood oximetry sensor) can be, for example, an optical sensor, an infrared sensor, a visible light sensor, or the like.

The electrical signal sensor can include two or more electrodes and can include circuitry to sense and record electrical signals including sensed electrical potentials and the magnitude thereof (according to Ohm's law where V=IR) as well as measure impedance from an applied electrical potential.

It will be appreciated that the sensor package can also include one or more sensors that are external to the ear-wearable device. In addition to the external sensors discussed hereinabove, the sensor package can comprise a network of body sensors (such as those listed above) that sense movement of a multiplicity of body parts (e.g., arms, legs, torso).

### Methods

Many different methods are contemplated herein, including, but not limited to, methods of making, methods of using, and the like. Aspects of system/device operation described elsewhere herein can be performed as operations of one or more methods in accordance with various embodiments herein.

In various embodiments, operations described herein and method steps can be performed as part of a computer-implemented method executed by one or more processors of one or more computing devices. In various embodiments, operations described herein and method steps can be implemented instructions stored on a non-transitory, computer-readable medium that, when executed by one or more processors, cause a system to execute the operations and/or steps.

In an embodiment, a method of providing vibration stimulation to an ear-wearable device wearer is included. The method can include evaluating signals from a sensor or a microphone to detect a trigger condition. The method can also include generating vibrations using a vibration generator when the trigger condition is detected.

In various embodiments, the sensor can include a touch sensor and the trigger condition can include the device wearer touching the touch sensor. In an embodiment, the method can further include evaluating signals from a touch sensor to evaluate signals from a touch sensor and generate the vibrations when a predetermined touch pattern is detected.

In an embodiment, the method can further include modulating a parameter of the vibrations based on a detected magnitude of device touching pressure.

In an embodiment, the method can further include delivering vibrations to a device wearer's fingertip contacting the vibration generator. In an embodiment, the method can further include delivering vibrations to a device wearer's external ear or ear canal contacting the vibration generator.

In some embodiments, the trigger condition can include an initiation sound. In an embodiment, the initiation sound can include at least one selected from the group consisting of a verbal command, an identified keyword or phrase, and an instability utterance.

In an embodiment, the trigger condition can include a motion sensor signal pattern. In an embodiment of the method, the motion sensor signal pattern is indicative of postural instability.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. An ear-wearable device comprising:
a control circuit;
a microphone, wherein the microphone is in electrical communication with the control circuit; and
a sensor package, the sensor package comprising a touch sensor;
wherein the sensor package is in electrical communication with the control circuit;
a vibration generator, wherein the vibration generator is in electrical communication with the control circuit;
wherein the ear-wearable device is configured to
evaluate signals from the touch sensor to detect device touching; and
generate vibrations using the vibration generator when device touching is detected.

2-3. (canceled)

4. The ear-wearable device of claim 1, further comprising an external housing;
wherein the touch sensor is disposed on the external housing; and
wherein the vibration generator is disposed on the external housing.

5. The ear-wearable device of claim 4, wherein the vibration generator and the touch sensor are co-located on the external housing.

6. The ear-wearable device of claim 1, wherein the vibration generator is configured to deliver vibrations to a device wearer's fingertip contacting the vibration generator.

7. The ear-wearable device of claim 1, wherein the vibration generator is configured to deliver vibrations to a device wearer's external ear or ear canal contacting the vibration generator.

8. The ear-wearable device of claim 1, wherein the ear-wearable device is configured to evaluate signals from the touch sensor to evaluate signals from the touch sensor and generate the vibrations when a predetermined touch pattern is detected.

9. The ear-wearable device of claim 1, wherein the vibrations have a frequency of 60 HZ to 1000 HZ.

10. The ear-wearable device of claim 1, wherein the vibrations have a frequency of 150 HZ to 350 HZ.

11. The ear-wearable device of claim 1, wherein the ear-wearable device is configured to modulate a parameter of the vibrations based on a detected magnitude of device touching pressure.

12. The car-wearable device of claim 11, the parameter comprising at least one selected from the group consisting of vibration frequency, vibration amplitude, and vibration duration.

13. The ear-wearable device of claim 1, the vibration generator comprising at least one selected from the group consisting of a piezoelectric vibration generator and an electromagnetic vibration generator.

14. The ear-wearable device of claim 13, the piezoelectric vibration generator comprising a piezoelectric crystal transducer.

15. The ear-wearable device of claim 1, the ear-wearable device comprising at least one selected from the group consisting of head phones, a RIC hearing aid, and a custom hearing aid.

16. An ear-wearable device comprising:
a control circuit;
a microphone, wherein the microphone is in electrical communication with the control circuit; and
a sensor package, the sensor package comprising a touch sensor;
wherein the sensor package is in electrical communication with the control circuit;
wherein the ear-wearable device is configured to
evaluate signals from the touch sensor to detect device touching; and
send a signal to a separate device to generate vibrations when device touching is detected.

17. An ear-wearable device comprising:
a control circuit;
a microphone, wherein the microphone is in electrical communication with the control circuit; and
a sensor package, the sensor package comprising a motion sensor;
wherein the sensor package is in electrical communication with the control circuit;
a vibration generator, wherein the vibration generator is in electrical communication with the control circuit;
wherein the ear-wearable device is configured to
evaluate signals from the motion sensor to detect postural instability; and
generate vibrations using the vibration generator when the postural instability is detected.

18. The ear-wearable device of claim 17, wherein the ear-wearable device is configured to generate vibrations using the vibration generator when postural instability exceeds a threshold value.

19. The ear-wearable device of claim 17, the postural instability comprising at least one selected from the group consisting of a leaning angle, a degree of sway, and stumbling.

20. The ear-wearable device of claim 17, further comprising an external housing, wherein the vibration generator is disposed on the external housing.

21-22. (canceled)

23. The ear-wearable device of claim 17, wherein the vibrations have a frequency of 60 HZ to 1000 HZ.

24. The ear-wearable device of claim 17, wherein the vibrations have a frequency of 150 HZ to 350 HZ.

25-53. (canceled)
